(19) United States
(12) Patent Application Publication
(10) Pub. No.: US 2013/0029998 A1
(43) Pub. Date: Jan. 31, 2013

(54) Method of Detecting and Profiling Progression of the Risk of Neurodegenerative Diseases

(71) Applicants: Chandra Shekhar Mayanil, Chicago, IL (US); Shunsuke Ichi, Chicago, IL (US); Tadanori Tomita, Chicago, IL (US)

(72) Inventors: Chandra Shekhar Mayanil, Chicago, IL (US); Shunsuke Ichi, Chicago, IL (US); Tadanori Tomita, Chicago, IL (US)

(21) Appl. No.: 13/640,545

(22) PCT Filed: Apr. 21, 2011
(86) PCT No.: PCT/US11/33387

§ 371 Date: Oct. 11, 2012

Related U.S. Application Data
(60) Provisional application No. 61/395,321, filed on May 11, 2010.

(51) Int. Cl. G01N 33/566; A61K 31/519; A61P 25/28; A61K 31/525

(52) U.S. Cl. 514/251; 435/7.1; 514/249

(57) ABSTRACT

The present invention generally relates to methods of detecting and profiling progression of the risk of neurodegenerative diseases in a subject. In one embodiment, the method includes isolating a stem cell from cerebrospinal fluid of the subject and determining the level of H3K27 methylation within the stem cell. The subject is determined to have an increased risk of developing the neurodegenerative disease if the level of H3K27 methylation is elevated. In various embodiments, the neurodegenerative disease is Alzheimer's Disease, Parkinson's Disease or Amyotrophic Lateral Sclerosis.

### TECHNICAL FIELD

The present invention generally relates to methods of detecting and profiling progression of the risk of neurodegenerative diseases and to methods of preventing and treating such diseases.

### BACKGROUND

Neurodegenerative disease is the term for the progressive loss of structure or function of neurons, including death of neurons as a result of a neurodegenerative processes. Such diseases including Parkinson's Disease, Alzheimer's Disease, and Amyotrophic Lateral Sclerosis (ALS.)

Alzheimer's disease is the most common form of dementia. Generally, it is diagnosed in people over 65 years of age, although the less-prevalent early-onset Alzheimer's can occur much earlier. In 2006, there were 26.6 million sufferers worldwide. Alzheimer's disease develops for an indeterminate period of time before the appearance of clinical symptoms and can remain undiagnosed for years. Although the course of Alzheimer's disease is unique for every individual, there are many common clinical symptoms. The earliest observable symptoms are often mistakenly thought to be age-related concerns, or manifestations of stress. In the early stages, the most commonly recognized symptom is memory loss, such as difficulty in remembering recently learned facts. As the disease advances, symptoms include confusion, irritability and aggression, mood swings, language breakdown, long-term memory loss and the general withdrawal of the patient. Eventually, bodily functions are lost, ultimately leading to death.

Parkinson's disease is a degenerative disorder of the central nervous system that often impairs the sufferer's motor skills, speech, and other functions. Its clinical symptoms are characterized by muscle rigidity, tremor, a slowing of physical movement and a loss of physical movement in extreme cases. The primary symptoms are the results of decreased stimulation of the motor cortex by the basal ganglia, normally caused by the insufficient formation and action of dopamine, which is produced in the dopaminergic neurons of the brain. Secondary clinical symptoms may include high level cognitive dysfunction and subtle language problems.

Amyotrophic lateral sclerosis (ALS or Lou Gehrig's disease) is a disease in which motor neurons are selectively targeted for degeneration. The onset of ALS may be so subtle that the clinical symptoms are frequently overlooked. The earliest clinical symptoms are obvious weakness and/or muscle atrophy. These symptoms are followed by muscle weakness and atrophy throughout the body as both the upper and lower motor neurons degenerate. Unable to function, the muscles gradually weaken, develop twitches and eventually atrophy. The patient may ultimately lose the ability to initiate and control all voluntary movement.

The first clinical symptoms of neurodegenerative diseases may occur many years after the onset of the disease. For example, in Parkinson's Disease, the majority of cell loss due to neurodegeneration occurs before the appearance of clinical symptoms. Early detection of the disease before clinical symptoms appear offers the promise of presymptomatic treatment and prevention, or at least a reduction, of cell loss. Accordingly, there is the need to develop a method of detecting a neurodegenerative disease in a patient early in the development of the disease, preferably before the onset of clinical symptoms.

### BRIEF SUMMARY

One aspect of the present invention provides a method of determining risk of developing a neurodegenerative disease in a subject. The method includes the steps of isolating a stem cell from cerebrospinal fluid of the subject and determining the level of H3K27 methylation within the stem cell. An elevated level of H3K27 methylation is indicative of an increased risk of developing the neurodegenerative disease. In one embodiment, the step of determining of the level of H3K27 methylation within the stem cell includes contacting the stem cell with antibody to at least one of dimethylated histone H3 at K27 and trimethylated histone H3 at K27. In another embodiment, the step of the determining of the level of H3K27 methylation within the stem cell includes contacting the stem cell with antibody specific to dimethylated histone H3 at K27 and trimethylated histone H3 at K27.

In various embodiments, the neurodegenerative disease is Alzheimer's Disease, Parkinson's Disease or Amyotrophic Lateral Sclerosis. In certain embodiments the subject is tested prior to having any clinical symptoms of the neurodegenerative disease.

In other embodiments, the method also includes determining the presence or absence in the stem cell of at least one of the CD133, Sox2, Oct4 or Nanog stem cell markers. In yet other embodiments, the method also includes determining the presence or absence in the stem cell of at least one differentiation marker selected from the group consisting of Brn3a, TuJ1, GFAP and O4.

In various embodiments, the method also includes determining the presence or amount of beta-amyloid protein in the stem cell by contacting the stem cell with beta-amyloid antibody or determining the presence or amount of alpha-synuclein protein in the stem cell by contacting the stem cell with alpha-synuclein antibody or determining the presence or amount of Interleukin-6 protein in the stem cell by contacting the stem cell with Interleukin-6 antibody.

Another aspect of the present invention generally relates to a method of monitoring the treatment of a subject at risk of a neurodegenerative disease. In one embodiment, the method includes isolating a stem cell from cerebrospinal fluid of the subject and determining the level of H3K27 methylation within the stem cell. The subject is determined to be at an increased risk of the neurodegenerative disease if the level of H3K27 methylation is higher than normal. The method also includes administrating a Vitamin B or a metabolite thereof to the subject if the subject is determined to be at an increased risk of the neurodegenerative disease. In one embodiment, the Vitamin B metabolite is L-methyl folate.

### BRIEF DESCRIPTION OF THE DRAWINGS

FIGS. 1(a)-1(c) are illustrations showing the reversal of H3K27 methylation and KDM6B expression in folate-rescued Splotch homozygous embryos. Appendix 1 includes a color illustration of these figures.

### DETAILED DESCRIPTION

### Early Detection of Neurodegenerative Diseases

One aspect of the present invention generally relates to methods of detecting and profiling progression of the risk of neurodegenerative diseases in a subject. In one embodiment the subject is a human subject. In various embodiments, the neurodegenerative disease is Alzheimer's Disease, Parkinson's Disease or Amyotrophic Lateral Sclerosis. Another aspect of the invention relates to methods of monitoring the treatment of a neurodegenerative disease in such subjects.

The adult brain has the capacity of neuro-restoration stemming mainly from the neural stem cells of sub-ventricular zone and the sub-granular zone. These stem cells undergo proliferation, migration differentiation and integration in various parts of the brain. In neurodegenerative disease this neurorestorative capacity is diminished and neurons are lost progressively.

Pax genes control certain aspects of cellular development. Mutations of these genes result in semi-dominant defects apparent during embryogenesis. The Pax-3 mutant mouse Splotch is a folate-responsive mouse model for spina bifida. The applicants have used the Splotch mouse model to demonstrate that Pax3 regulates the Hes1 and Neurog2 genes [Nakazaki et al. Development Biology 316, pp. 510-523 (2008)]. Splotch homozygous embryos exhibit an open neural tube defect and have an increase in Histone 3 methylation at position 27 and a decrease in KDM6B, a histone demethylase.

Histones are strongly alkaline proteins found in eukaryotic cell nuclei that package the DNA. Histones can be grouped into five major classes: H1/H5, H2A, H2B, H3, and H4. These classes are organized into two super-classes: core histones—H2A, H2B, H3 and H4 and linker histones—H1 and H5.

Histones undergo posttranslational modifications which alter their interaction with DNA and nuclear proteins. The histones can be covalently modified at several places by, for example methylation, acetylation and phosphorylation. Histone modifications act in diverse biological processes such as gene regulation, DNA repair and chromosome condensation during mitosis. The common nomenclature of histone modifications is the name of the histone followed by the single letter amino acid abbreviation of the amino acid modified and the amino acid position in the protein. Finally, the type of modification is indicated. For example, H3K27me2 denotes the dimethylation of the 27th residue (a lysine) from the N-terminal of the H3 protein.

The Hes 1 and Neurog 2 genes are involved in stem cell maintenance and neurogenesis. It is believed that decreased expression levels of these genes may be due to increased H3K27me levels on Hes1 and Neurog 2 promoters. Elevated H3K27me levels in neural stem cells are believed to be an indicator of a reduced ability of stem cells to proliferate, resulting in progressive loss of neurons and eventual neurodegeneration.

One aspect of the present invention provides a method of detecting the risk of development of a neurodegenerative disease in a subject by determining the level of H3K27 methylation in a neural stem cell isolated from the cerebrospinal fluid of the subject. An elevated level of H3K27 methylation is indicative of an increased risk of neurodegenerative disease. Another aspect of the present invention provides a method of detecting the risk of a neurodegenerative disease by determining the level of KDM6B in such a neural stem cell. A below normal level of KDM6B is indicative of an increased risk of neurodegenerative disease. In one embodiment, the method of detecting the risk of development of a neurodegenerative disease is a immunostaining method.

In one embodiment, the subject is a human subject. In certain embodiments, the risk of the neurodegenerative disease is detected prior to the human subject showing any clinical symptoms of the disease. In various embodiments, the risk of neurodegenerative disease is detected at least 15 years, 10 years, 5 years or one year prior to the onset of clinical symptoms.

In certain embodiments, the stem cell is identified as a cell positive for at least one of the stem cell markers: CD133, Sox2 (an HMG box transcription factor), Oct4 (Octamer-4) or Nanog (NANOG protein). Preferably the stem cell is an undifferentiated stem cell. In certain embodiments, the undifferentiated stem cell is a stem cell that is negative for at least one of the differentiation markers: Brn3a (Brain-specific homeobox/POU domain protein 3A), TuJ1 (Neuron-specific class III beta-tubulin), GFAP (glial fibrillary acid protein) and O4 (oligodendrocytes marker.)

In one embodiment, the nuclear stain 4′,6-diamidino-2-phenylindole (DAPI) is contacted with the cell. When added to a cell, DAPI will stain the DNA blue. In other embodiments, antibodies to at least one of the other markers mentioned are labeled with a detectable probe and contacted with the cell. For example, mouse monoclonal antibodies to at least one of these markers are labeled with a fluorescent compound.

The labeled antibodies are contacted with the cell. The presence or absence of a particular marker is determined by observing whether the labeled antibody binds to the cell by detecting the presence of the detectable probe in the cell. In one embodiment, antibodies specific to different markers are labeled with different fluorescent compounds having differing absorption or emission spectra, allowing for the detection of multiple markers in the same cell.

One embodiment of the present invention provides a method including isolating an undifferentiated neural stem cell from cerebrospinal fluid of the subject and determining the level of H3K27 methylation within the stem cell. The subject is determined to have an increased risk of developing a neurodegenerative disease if the level of H3K27 methylation within the undifferentiated neural stem cell is elevated. In one embodiment, the subject is determined to have an increased risk of developing the neurodegenerative disease if the level of H3K27 di-methylation is elevated. In another embodiment, the subject is determined to have an increased risk of developing the neurodegenerative disease if the level of H3K27 tri-methylation is elevated. In yet another embodiment, the subject is determined to have an increased risk of developing the neurodegenerative disease if the level of H3K27 tri-methylation and/or di-methylation is elevated.

In one embodiment, the level of H3K27 methylation within the stem cell is determined by a method including by contacting the stem cell with an antibody specific to at least one of dimethylated histone H3 at K27 and trimethylated histone H3 at K27. The antibody may be a monoclonal antibody or a polyclonal antibody. In certain embodiments, the antibody is labeled with a detectable probe, for example, a radioactive probe, a fluorescent probe or a chemiluminescent probe. In other embodiments, the antibody is not labeled with a probe. Instead, the presence of the antibody is detected by contacting the antibody with a secondary antibody labeled with such a detectable probe.

In another embodiment, the level of H3K27 methylation within the stem cell in determined in combination with a determination of the cerebrospinal fluid level of homocysteine. Studies have shown that elevated levels of homocysteine, an amino acid not incorporated in protein synthesis, is a major risk factor in neurodegenerative disease etiology. Troen, A. M. et al., Proc. Natl. Acad. Sci. U.S.A., 105(34), pp. 12474-9 (2008.) In one embodiment, an elevated level of H3K27 methylation in combination with an elevated cerebrospinal fluid level of homocysteine is indicative of an increased risk of the neurodegenerative disease. In another embodiment, an elevated level of H3K27 methylation in combination with a level cerebrospinal fluid homocysteine within the normal range is indicative of an early stage of disease etiology.

In other embodiments, a determination of the level of H3K27 methylation is performed in combination with a secondary test to categorize the type of neurodegenerative disease the subject is at risk of developing. In one embodiment, the presence or amount of beta-amyloid protein or transforming growth factor beta 1 (TGFbeta 1) in the stem cell is determined, for example, by a method including contacting the stem cell with beta-amyloid or TGFbeta 1 antibody. In this embodiment, the presence or an increased amount of beta-amyloid protein or TGFbeta 1 is indicative of an increased risk of developing Alzheimer's Disease.

In another embodiment, the presence or amount of alpha-synuclein protein or p-tau in the stem cell is determined, for example, by a method including contacting the stem cell with alpha-synuclein antibody or p-tau antibody. In this embodiment, the presence or increased amount of beta-amyloid protein or p-tau is indicative of an increased risk of developing Parkinson's Disease.

In yet another embodiment, the presence or amount of Interleukin-6 or pigment epithelium derived factor (PEDF) in the stem cell is determined, for example, by contacting the stem cell with Interleukin-6 antibody or PEDF antibody. In this embodiment, the presence or increased amount of Interleukin-6 or PEDF is indicative of an increased risk of developing ALS.

In another embodiment, the risk of a neurodegenerative disease is determined measuring the presence or amount of KDM6B present in a neural stem cell by a method including contacting the neural stem cell with an antibody to KDM6B. In one embodiment, the method is an immunostaining method. A below normal level of KDM6B is indicative of an increased risk of developing an neurodegenerative disease.

### Monitoring the Treatment of a Neurodegenerative Disease

Another aspect of the present invention provides a method of preventing the development of or monitoring the treatment of a neurodegenerative disease. In one embodiment, a subject having an elevated level of H3K27 methylation within a neural stem cell is treated with a Vitamin B or a metabolite thereof, for example, the biologically active folate form L-methylfolate. The level of H3K27 methylation is measured at intervals during the treatment process. A decrease in the level of H3K27 methylation as treatment progresses is indicative of a decreased risk of developing a neurodegenerative disease.

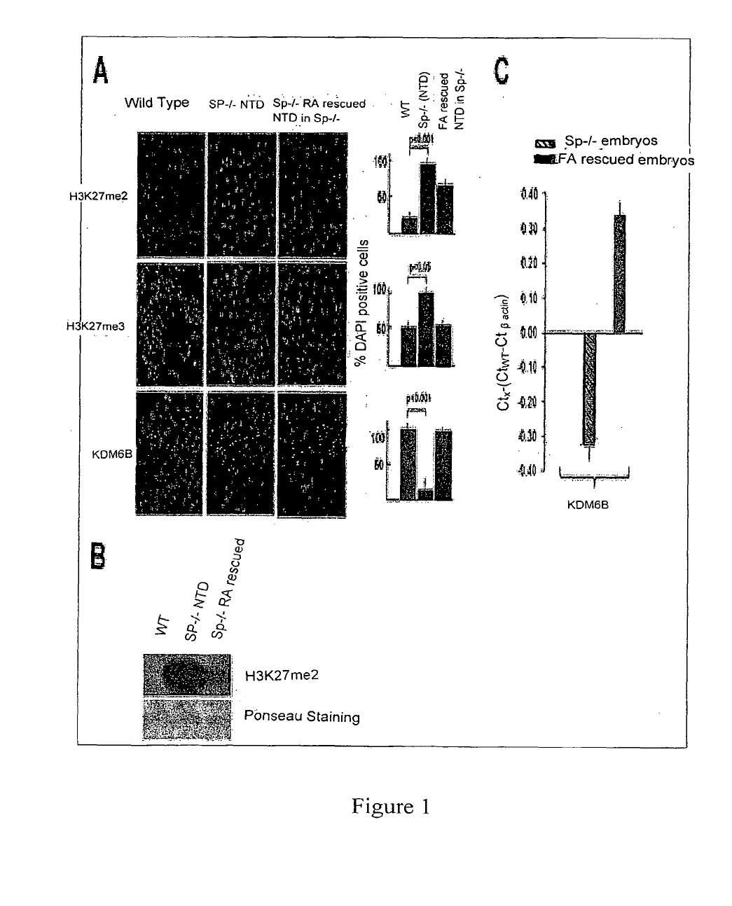

The reversal of the marker H3K27me2 by folic acid in splotch embryos and rescue of neural tube defect is illustrated in the Pax3 mutant Splotch-folic acid responsive mouse model of spina bifida. Splotch homozygous embryos exhibit an open neural tube defect and have an increase in H3K27 methylation and a decrease in KDM6B. FIG. 1 shows the reversal of H3K27 methylation and KDM6B expression in folate-rescued Splotch homozygous embryos. FIG. 1(A) shows neural tube explants from the lower lumbar neural tube region of E10.0 (30 somite) wild-type and Sp−/− and folate-rescued Sp−/− embryos initially grown in the presence of the growth factors EGF and bFGF. After 140 hours neural crest cells were allowed to differentiate in the absence of growth factor for 2 days. After differentiation, neural crest cells from wild-type and Sp−/− embryo explant cultures were stained with DAPI, H3K27me2, H3K27me3 and KDM6B antibodies (Abeam, Inc Cambridge, Mass. 02139-1517). DAPI, H3K27me2, H3K27me3 and KDM6B positive cells from five different explant cultures were counted and expressed as a percentage of DAPI positive cells. H3K27me2 (p<0.001) and H3K27me3 (p<0.05) positive staining was significantly increased and KDM6B staining significantly decreased (p<0.001) in Sp−/− embryo neural tube explants as determined by Student's T-test.

Folic acid-rescued Sp−/− embryos showed reversal to near wild-type expression of H3K27me2, H3K27me3 and KDM6B. FIG. 1(B) shows an H3K27me2 immunoblot of acid extracted histones from closed caudal neural tubes of wild-type, open caudal neural tube of Sp−/−, and closed caudal neural tube of folate-rescued Sp−/− embryos (E10.0; 30 somite). The protein loading control was ascertained with Ponseau S staining.

FIG. 1(C) shows murine KDM6B expression analyzed by RT-PCR using the Applied BioSystem 7500 Fast Real-Time thermal cycler. The data (n=4; mean+S.E.M) shows ΔΔCt values for KDM6B transcript levels in Sp−/−, folate-rescued Sp−/− embryos compared to wild type littermates. β-actin was used as a positive control. The data is normalized to β-actin levels. ΔΔCt value is obtained by subtracting the Ct value of the wild-type KDM6B from Sp−/− or folate-rescued Sp−/− after normalization with β-actin. A ΔΔCt value close to zero is more close to wild-type gene expression. A ΔΔCt value greater than zero indicates higher gene expression and values less than zero indicates lower expression than wild-type. One cycle change in ΔΔCt value indicates a 2 fold change in the gene expression. Experiments were performed in quadruplicate with each data point in duplicate.

### Kits and Reagents for Detecting and Profiling Progression of the Risk of Neurodegenerative Diseases

The present invention also provides for kits and reagents for detecting and profiling progression of the risk of neurodegenerative diseases. In one embodiment, the kit contains at least a reagent comprising an antibody specific to at least one of dimethylated histone H3 at K27 and trimethylated histone H3 at K27 in combination with a buffer in a package or container. The antibody may be a monoclonal antibody or a polyclonal antibody. In certain embodiments, the antibody is labeled with a detectable probe, for example, a radioactive probe, a fluorescent probe or a chemiluminescent probe. In other embodiments, the antibody is not labeled with a probe. Instead, the kit further includes a secondary antibody labeled with such a detectable probe. In these embodiments and in the embodiments described below the buffer can be in a liquid, frozen or a freeze dried form.

In certain embodiments, the kit also includes a reagent including a stem cell marker in combination with a buffer in a package or container. Examples of such markers include but are not limited to: CD 33, Sox2, Oct4 or Nanog. In other embodiments, the kit also includes a reagent including a marker of stem cell differentiation in combination with a buffer in a package or container. Examples of such markers include but are not limited to: Brn3a (sensory neuron marker), TuJ1 (neuronal marker), GFAP (astrocytes marker) and O4 (oligodendrocytes marker.) In another embodiment, the kit also includes a reagent including a nuclear stain, such as DAPI, in combination with a buffer in a package or container.

In other embodiments, the kit includes one or more wash buffers, (for example, Phosphate buffered saline) and/or blocking buffers (for example, 5% Normal Donkey Serum/0.01% Triton X-100/0.01% sodium azide in PBS) in packages or containers. In yet other embodiments, the kits may include a signal generation reagent for development of a detectable signal from the signaling moiety. The kits may also include one or more sample collection devices, for example a syringe or needle suitable for performing a lumbar puncture. In other embodiments, the kits also include positive and/or negative control samples in suitable packages or containers.

When a kit is supplied, the different components may be packaged in separate containers and admixed immediately before use. Such packaging of the components separately may permit long-term storage without losing the active components' functions. Kits may also be supplied with instructional materials. Instructions may be printed on paper or other substrate, and/or may be supplied as an electronic-readable medium, such as a floppy disc, CD-ROM, DVD-ROM, Zip disc, videotape, and audiotape. Detailed instructions need not be physically associated with the kit; instead, a user may be directed to an Internet web site specified by the manufacturer or distributor of the kit, or supplied as electronic mail.

### EXAMPLES

### Prophetic

### Example 1

### Immunochemical Detection of H3K27 Methylation in Neural Stem Cells

A sample of cerebrospinal fluid is obtained from a subject using lumbar puncture. The cells in the sample are separated and washed three times with Phosphate buffered saline, pH 7.5 (PBS). The cells are coated onto a glass slide and fixed by incubation for 10 minutes in 4% paraformaldehyde (PFA) in PBS. The cells are then again washed three times in PBS.

A blocking solution (5% Normal Donkey Serum/0.01% Triton X-100/0.01% sodium azide in PBS) is added for at least one hour. The blocking solution is removed and primary rabbit antibodies to H3K27 methylated (Abeam, Inc Cambridge, Mass. 02139-1517) diluted as per the manufacturer's instructions in the blocking solution is added. The cells are incubated overnight with antibody solution at 4° C. The antibody solution is removed and the cells washed three times in PBS. A solution of donkey-anti-rabbit secondary antibodies conjugated with horseradish peroxidase and diluted as per the manufacturer's instructions in the blocking solution is added and the cells incubated for one hour. The secondary antibody solution is removed and the cells again washed three times in PBS. A substrate solution is added and the presence or amount of H3K27 methylation determined by measuring the amount of color development.

### Example 2

### Immunochemical Detection of H3K27 Methylation in Undifferentiated Neural Stem Cells

Cells are isolated from the cerebrospinal fluid of a subject as in Example 1. The isolated cells are contacted with a first monoclonal antibody raised against a CD133, Sox2, Oct4 or Nanog stem cell marker. The first monoclonal antibody is labeled with a first fluorescent probe having a first frequency emission range. The isolated cells are also contacted with at a second monoclonal antibody raised against a Brn3a, TuJ1, GFAP or O4 differentiation marker. The second monoclonal antibody is labeled with a second fluorescent probe having a second emission frequency range that is detectable in the presence of the first fluorescent probe. In addition, the cells are contacted with an antibody raised against H3K27 methylated labeled with a third fluorescent probe that is detectable in the presence of the first and second fluorescent probes.

Undifferentiated stem cells are identified as those cells binding the first monoclonal antibody but not the second monoclonal antibody. The level of H3K27 methylation present in these cells is determined by measuring the amount of the third fluorescent probe.

### Example 3

### Early Detection Test for Risk of Alzheimer's Disease

A sample of cerebrospinal fluid is obtained from a subject and the cells contained in the sample washed and fixed using the protocol described in Example 1. The cells may also be incubated in Formic acid/pH 1.6-2.0 at room temperature for 10-20 minutes to improve antigen retrieval.

A mouse monoclonal antibody to human beta amyloid protein is added for 60 minutes at room temperature. (Clone: 6F/3D, Mouse Anti-Human, Novocastra Labs, Catalog Number: NCL-B-Amyloid.) The antibody is diluted 1:100 using IHC-Tek™ Antibody Diluent (Cat#1W-1000 or 1W-1001) to reduce background and nonspecific staining. A separate serum blocking step using Normal Donkey Serum is not needed. Endogenous peroxidase activity is then blocked by incubating the cells 0.3% H₂O₂.

After blocking, the cells are incubated in biotinylated secondary antibody in PBS for 30 minutes at room temperature. After washing away unbound antibody, the presence of the bound biotinylated antibody is detected using an ImmunoPure ABC Staining Kit (Thermo Fisher Scientific, Rockford Ill. 61105.) Increased risk of Alzheimer's Disease is indicated by the excessive presence of beta amyloid protein in senile plaque cores, plaque periphery and diffuse plaques.

### Example 4

### Early Detection Test for Risk of Parkinson's Disease

The basic procedure is as in Example 3 except that the primary antibody a mouse anti-α-synuclein monoclonal antibody, Clone: KM51 (Novocastra Laboratories Newcastle upon Tyne NE12 8EW UK). Increased risk of Parkinson's Disease is indicated by the excessive presence of Lewy body and neuropil staining.

### Example 5

### Early Detection Test for Risk of ALS

The basic procedure is as in Example 3 except that the primary antibody a rabbit IL-6 polyclonal antibody, Catalog Number: ab6672 (Abeam, Inc Cambridge, Mass. 02139-1517). An elevated level of IL-6 is indicative of increased risk of ALS.

Although the invention has been described and illustrated with reference to specific illustrative embodiments thereof, it is not intended that the invention be limited to those illustrative embodiments. Those skilled in the art will recognize that variations and modifications can be made without departing from the true scope and spirit of the invention as defined by the claims that follow. It is therefore intended to include within the invention all such variations and modifications as fall within the scope of the appended claims and equivalents thereof.

We claim:

1. A method of determining risk of developing a neurodegenerative disease in a subject, the method comprising:
isolating a stem cell from cerebrospinal fluid of the subject;
determining the level of H3K27 methylation within the stem cell,
wherein an elevated level of H3K27 methylation is indicative of an increased risk of developing the neurodegenerative disease.

2. The method of claim 1, wherein the determining of the level of H3K27 methylation within the stem cell comprises contacting the stem cell with antibody to at least one of dimethylated histone H3 at K27 and trimethylated histone H3 at K27.

3. The method of claim 2, wherein the determining of the level of H3K27 methylation within the stem cell comprises contacting the stem cell with antibody specific to at least one of dimethylated histone H3 at K27 and trimethylated histone H3 at K27.

4. The method of claim 1, where the neurodegenerative disease is selected from the group consisting of Alzheimer's Disease, Parkinson's Disease and Amyotrophic Lateral Sclerosis.

5. The method of claim 4, wherein the neurodegenerative disease is Alzheimer's Disease.

6. The method of claim 4, wherein the neurodegenerative disease is Parkinson's Disease.

7. The method of claim 4, wherein the neurodegenerative disease is Amyotrophic Lateral Sclerosis.

8. The method of claim 1, further comprising determining the presence or absence in the stem cell of at least one stem cell marker selected from the group selected consisting of CD133, Sox2, Oct4 and Nanog, wherein the determining of the stem cell marker comprises contacting the stem cell with an antibody to at least one of CD133, Sox2, Oct4 and Nanog.

9. The method of claim 8, wherein the stem cell is positive for at least one stem cell marker selected from the group selected consisting of CD133, Sox2, Oct4 and Nanog.

10. The method of claim 1, further comprising determining the presence or absence in the stem cell of at least one differentiation marker selected from the group consisting of Brn3a, TuJ1, GFAP and O4, wherein the determining of the differentiation marker comprises contacting the stem cell with an antibody to at least one of Brn3a, TuJ1, GFAP and O4.

11. The method of claim 10, therein the stem cell is negative for at least one differentiation marker selected from the group consisting of Brn3a, TuJ1, GFAP and O4.

12. The method of claim 1, further comprising determining the presence or amount of beta-amyloid protein in the stem cell by contacting the stem cell with beta-amyloid antibody.

13. The method of claim 1, further comprising determining the presence or amount of alpha-synuclein protein in the stem cell by contacting the stem cell with alpha-synuclein antibody.

14. The method of claim 1, further comprising determining the presence or amount of Interleukin-6 protein in the stem cell by contacting the stem cell with Interleukin-6 antibody.

15. The method of claim 1, wherein the subject is tested prior to having any clinical symptoms of the neurodegenerative disease.

16. A method of treating a subject at risk of a neurodegenerative disease, the method comprising:
isolating a stem cell from cerebrospinal fluid of the subject;
determining the level of H3K27m2 methylation within the stem cell, wherein the subject is determined to be at an increased risk of the neurodegenerative disease if the level of H3K27 methylation is higher than normal, and
administrating a Vitamin B or a metabolite thereof to the subject if the subject is determined to be at an increased risk of the neurodegenerative disease.

17. The method of claim 16, wherein the Vitamin B metabolite is L-methyl folate.

18. The method of claim 16, further comprising determining the presence or absence in the stem cell of at least one stem cell marker selected from the group selected consisting of CD133, Sox2, Oct4 and Nanog, wherein the determining of the stem cell marker comprises contacting the stem cell with an antibody to at least one of CD133, Sox2, Oct4 and Nanog.

19. The method of claim 16, further comprising determining the presence or absence in the stem cell of at least one differentiation marker selected from the group consisting of Brn3a, TuJ1, GFAP and O4, wherein the determining of the differentiation marker comprises contacting the stem cell with an antibody to at least one of Brn3a, TuJ1, GFAP and O4.
